(19) United States
(12) Patent Application Publication
(10) Pub. No.: US 2008/0108898 A1
(43) Pub. Date: May 8, 2008

(54) METHOD FOR PRODUCING MODEL OF FIBROUS STRUCTURE OF FIBROUS TISSUE

(76) Inventors: Ali MEGHOUFEL, Longueuil (CA); Jacques A. De Guise, Montreal (CA); Guy Cloutier, Le Gardeur (CA)

Correspondence Address: OGILVY RENAULT LLP, 1981 MCGILL COLLEGE AVENUE, SUITE 1600, MONTREAL, QC, H3A2Y3, CA

(21) Appl. No.: 11/867,234

(22) Filed: Oct. 4, 2007

Related U.S. Application Data
(60) Provisional application No. 60/828,141, filed on Oct. 4, 2006.

(51) Int. Cl. A61B 8/00

(52) U.S. Cl. 600/443

(57) ABSTRACT

A method for producing models of fibrous structure of fibrous tissue comprises obtaining a sequence of two-dimensional ultrasound images along the fibrous tissue. A three-dimensional model of an external portion of the fibrous tissue is created using the two-dimensional ultrasound images. Selected fibrous structure data is segmented from the two-dimensional ultrasound images. A three-dimensional model of the fibrous tissue is created with the fibrous structure by combining the three-dimensional model of the external portion of the fibrous tissue with the selected fibrous structure data.

### CROSS-REFERENCE TO RELATED APPLICATION

This patent application claims priority on U.S. Provisional Patent Application No. 60/828,141, filed on Oct. 4, 2006.

### FIELD OF THE APPLICATION

The present application relates to in vivo ultrasound imaging and more particularly to the creation of models of fibrous tissues such as tendons and ligaments from two-dimensional ultrasound images.

### BACKGROUND ART

Ultrasonic examination is a widely used diagnostic technique to evaluate the fibrous tissue, as is described by Crevier-Denoix, N., et al. in Correlations between mean echogenicity and material properties of normal and diseased equine superficial digital flexor tendons: an in vitro segmental approach, Journal of Biomechanics, 2005, 38(11), pp. 2212-2220. For instance, equine tendon structures must often be evaluated after an injury and during the healing process using ultrasound examination. In such cases, the equine superficial digital flexor tendon (SDFT) is a frequently injured structure. The required high physical performances are causing an increase and a diversification of this pathology.

Tendons are organized into a hierarchy of structures that include collagen (main structural protein), fibrils, fibers and fascicles (Schie, H. T. M. V., et al. Ultrasonographic tissue characterization of equine superficial digital flexor tendons by means of gray level statistics, American Journal of Veterinary Research, 2000, 61(2), pp. 210-219, and Garcia, T., W. J. Hornof, and M. F. Insana, On the ultrasonic properties of tendon, Ultrasound in Medicine and Biology, 2003, 29(12), pp. 1787-1797). A SDFT is shown at 1 in FIG. 1, with fibers arranged in primary fiber bundles 2 (subfascicles) of collagen fibers 2A divided in collagen fibrils 2B. These bundles gather to form secondary fiber bundles 3 (fascicles), and these latter bundles 3 are joined together in the same bundles called tertiary fiber bundles 4. The tertiary fiber bundles 4 are surrounded by loose connective tissue, called the endotendon 5 or interfascicular connective tissue, which provides vascular supply, lymphatic vessels, and probably a circular support, although the fascicles 3 are regularly parallel to the axis of the tendon 1.

On transverse two-dimensional ultrasound images (hereinafter 2D US images), healthy fibrous tissues such as SDFTs appear parallel and as linear hyper-echoic structures (Martinoli, C., et al., Analysis of Echotexture of Tendons with Us, Radiology, 1993, 186(3), pp. 839-843). These echoes are caused by the coherent specular reflections at the interfascicular, which are perpendicular to the US beam. In injured SDFTs, areas where fibers are disrupted appear as hypo-echoic structures, due to the disorganization of the interfascicular and the loss in collagen density.

Several studies such as those described above and by Crevier-Denoix, N., et al., in Mechanical correlations derived from segmental histologic study of the equine superficial digital flexor tendon, from foal to adult, American Journal of Veterinary Research, 1998, 59(8), pp. 969-977, were conducted to understand the internal tendon structure, to document injuries and to evaluate the integrity of fibrous tissue such as the SDFT. The most known methods use histological correlation in vitro, which is based on a comparison between transverse 2D US images matched with corresponding histological sections, However, these techniques are limited because it is difficult to implement correctly the method and to analyse the information contained on both images.

### SUMMARY OF APPLICATION

It is therefore an aim of the present application to provide a novel imaging method to evaluate the internal fibrous structure of fibrous tissues such as tendons and ligaments.

Therefore, in accordance with the present application, there is provided a method for producing models of fibrous structure of fibrous tissue, comprising the steps of: obtaining a sequence of two-dimensional ultrasound images along a fibrous tissue; creating a three-dimensional model of an external portion of the fibrous tissue using the ultrasound images; segmenting selected fibrous structure data from the two-dimensional ultrasound images; and creating a three-dimensional model of the fibrous tissue with the fibrous structure by combining the three-dimensional model of the external portion of the fibrous tissue with the selected fibrous structure data.

### BRIEF DESCRIPTION OF THE DRAWINGS

FIG. 1 is a perspective enlarged view of an internal fibrous structure of a superficial digital flexor tendon (SDFT);

FIG. 2 is a two-dimensional ultrasound image (2D US image) illustrating a limb with a healthy SDFT;

FIG. 3 is the 2D US image of FIG. 2, with a perimeter of the SDFT being defined;

FIG. 4 is a three-dimensional (3D) model of the SDFT resulting from the combination of 2D US images such as that of FIG. 3;

FIG. 5 is the 3D model of the SDFT of FIG. 4, having been subjected to a correction;

FIG. 6A is the 2D US image of FIG. 2, with a perimeter of the SDFT being defined;

FIG. 6B is the 2D US image of FIG. 6A, showing the echo signal envelope;

FIG. 6C is the 2D US image of FIG. 6B, after blind deconvolution;

FIG. 6D is the 2D US image of FIG. 6C, showing open fibrous structures;

FIG. 6E is the 2D US image of FIG. 6D, with closed fibrous structures;

FIG. 6F is the 2D US image of FIG. 6E as combined with the 2D US image of FIG. 6A;

FIG. 7 is a 3D model of the SDFT as produced with a method in accordance with an embodiment of the present application, showing fibrous structure obtained from the combination of 2D US images of FIG. 6E and the 3D model of FIG. 5;

FIG. 8 is a 2D model of an injured SDFT equivalent to the 2D US image of FIG. 6E;

FIG. 9 is a 3D model of the injured SDFT of FIG. 8, as produced with the method of the present application;

FIG. 10 is a block diagram of the method for producing models of fibrous structures of fibrous tissue in accordance with an embodiment of the present application;

FIG. 11A is a graph showing a number distribution of fiber bundles on SDFT cross-sectional US images obtained using the method of the present application; and

FIG. 11B is a graph showing an area distribution of fiber bundles on SDFT cross-sectional US images obtained using the method of the present application.

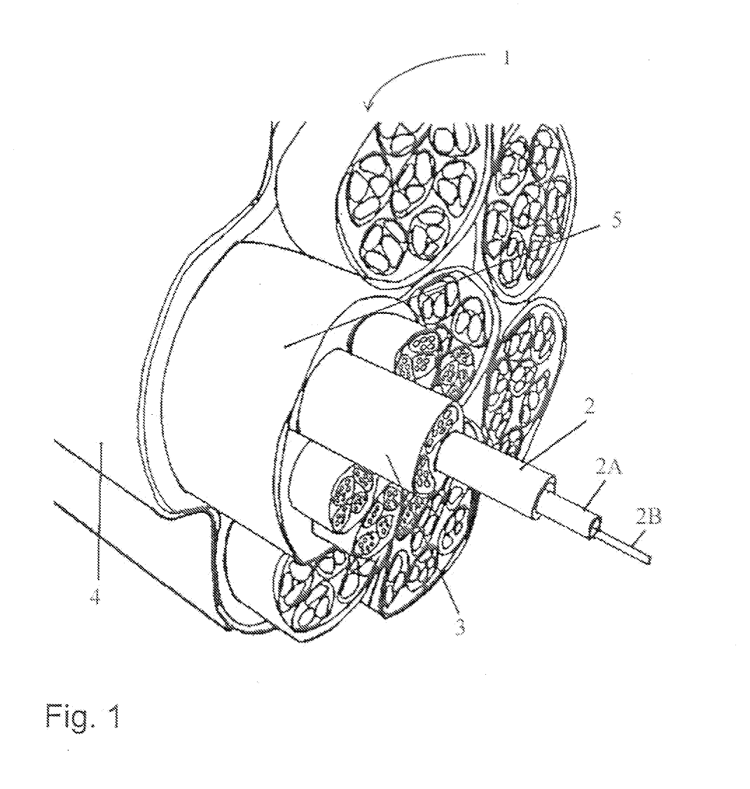

### DESCRIPTION OF THE PREFERRED EMBODIMENTS

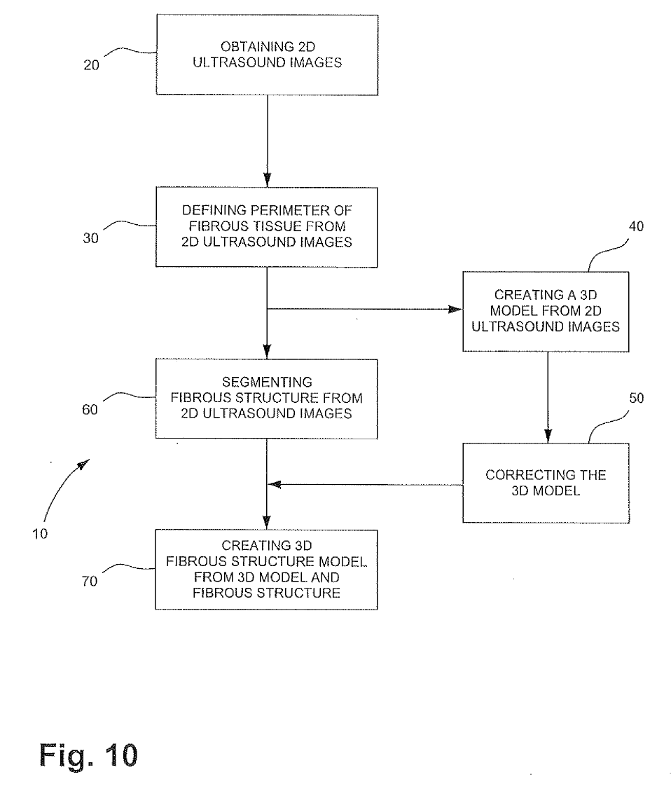

Referring to the drawings and more particularly to FIG. 10, an in vivo method for producing models of internal fibrous structures of fibrous tissue is generally illustrated at 10. The method is described as being used with healthy and injured SDFTs as an example, but is also used for imaging various animal and/or human fibrous tissues such as tendons and ligaments.

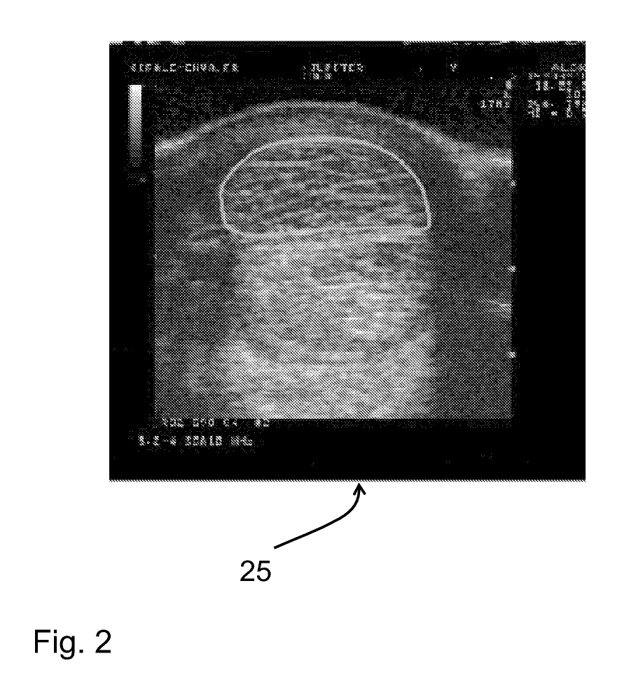

In Step 20 of the method 10, 2D US images are obtained. In an embodiment, the 2D US images are obtained in vivo from a healthy and an injured SDFT in freehand mode scanning. As an example, a 7.5 MHz linear array transducer (SSD-2000-7.5, Aloka) is used in Step 20. As a result, a plurality of 2D US images 25 are obtained such as the image illustrated in FIG. 2, representing slices/frames at incremental heights of the limb being scanned.

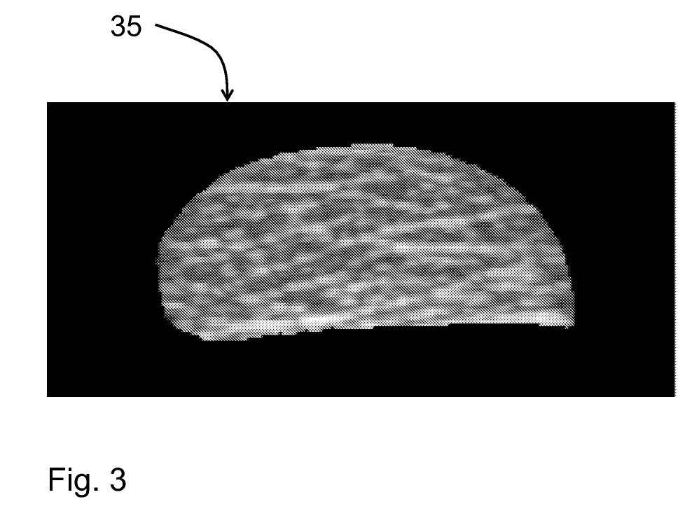

In Step 30 of the method 10, the perimeter/boundary of the fibrous tissue to be modeled is defined for each 2D US image. As shown in FIG. 3, the SDFT region 35 is identified and outlined, and this is performed for each successive frame.

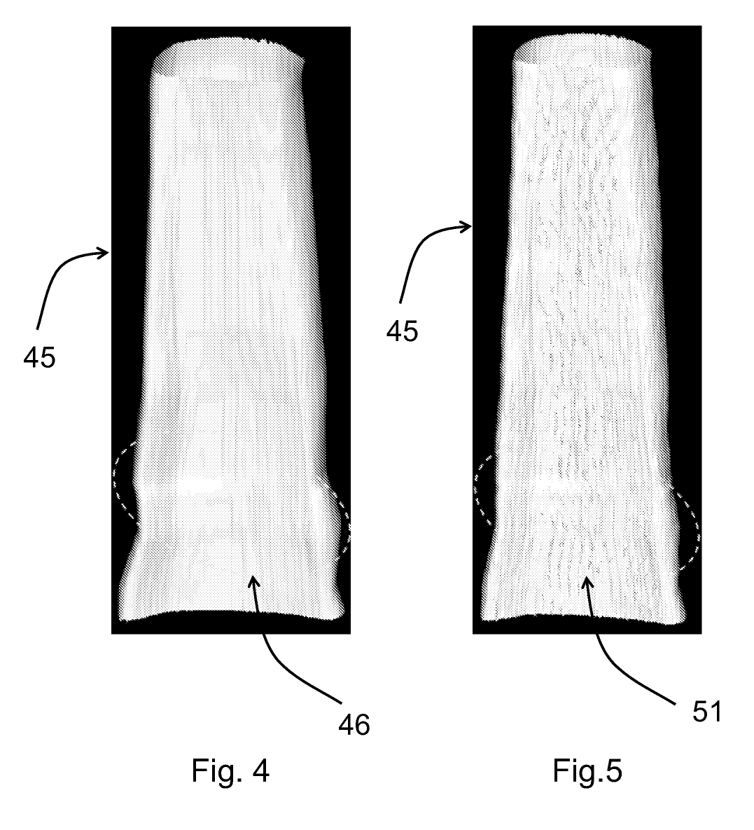

In Steps 40 and 50 of the method 10, a 3D digital model 45 of the external structure of the fibrous tissue is created. In Step 40, an initial 3D model 45 is created by merging the sequence of frames of 2D US images 35 defined in Step 20. The 3D model 45 of FIG. 4 is therefore obtained.

In one embodiment, the 3D model is potentially deformed as a result of hand movement during free-hand mode scanning. Accordingly, in Step 50, a correction is made to the 3D model 45 to reduce the effect of the hand movement on the 3D model 45. In an embodiment, alignment by 2D rigid body registration is used to correct the 3D model. By definition, the 2D contour/perimeter is the closed curve that surrounds the fibrous tissue (i.e., SDFT) in each cross sectional frame. Matching of two sequential 2D US images is performed by finding the 2D rotations R and translations T between two 2D contours that optimize a mutual function, as set forth in Thevenaz, P., U. E. Ruttimann, and M. Unser, A pyramid approach to subpixel registration based on intensity, Image Processing, IEEE Transactions on, 1998, 7(1), p. 27-41. The centroids Bₛ and B_{c} of the 2D contour source (Sᵢ) and contour target (Cᵢ) points are respectively computed. The translation vector is defined as T=Bₛ\vec{B}_{C}. The rotation R is estimated through the minimization of the following criterion:
R*=argmin∥Cᵢ−R(T(Sᵢ))∥² (1)
where R* is the iterated estimation of R and ∥·∥ is the Euclidean distance. Each 2D US image is used as the template to which the sequentially following 2D US image is aligned. The alignment is thus propagated through the image series.

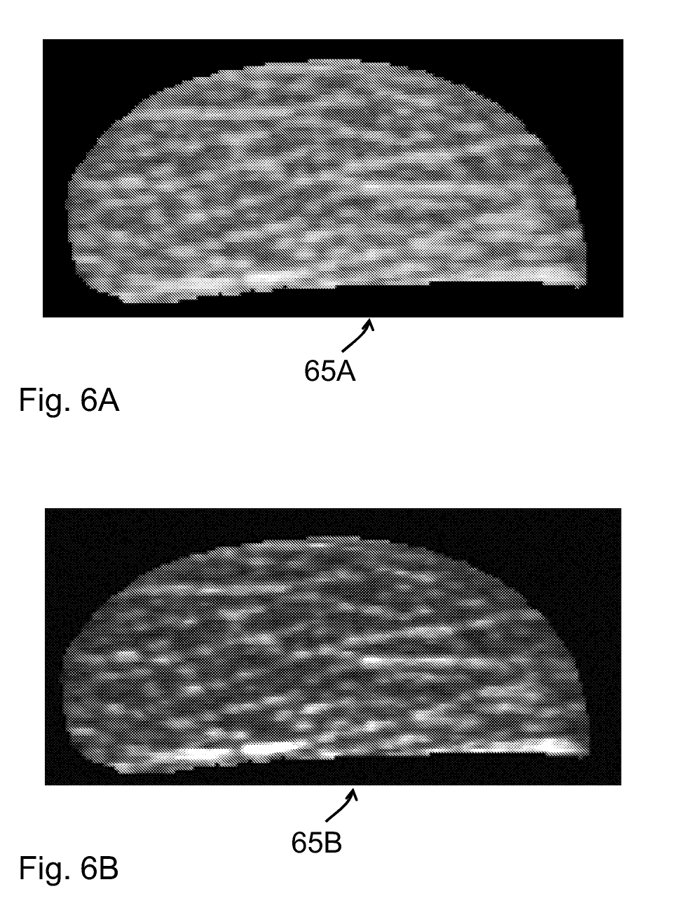

Referring concurrently to FIGS. 4 and 5, there is shown the 3D model of the fibrous tissue resulting from Step 40 (FIG. 4), and the corrected 3D model resulting from Step 50 (FIG. 5). The volume distortion 46, which is located at the bottom of the SDFT (FIG. 4), is corrected by this transformation. It results in a more coherent volume 51 shown in FIG. 5.

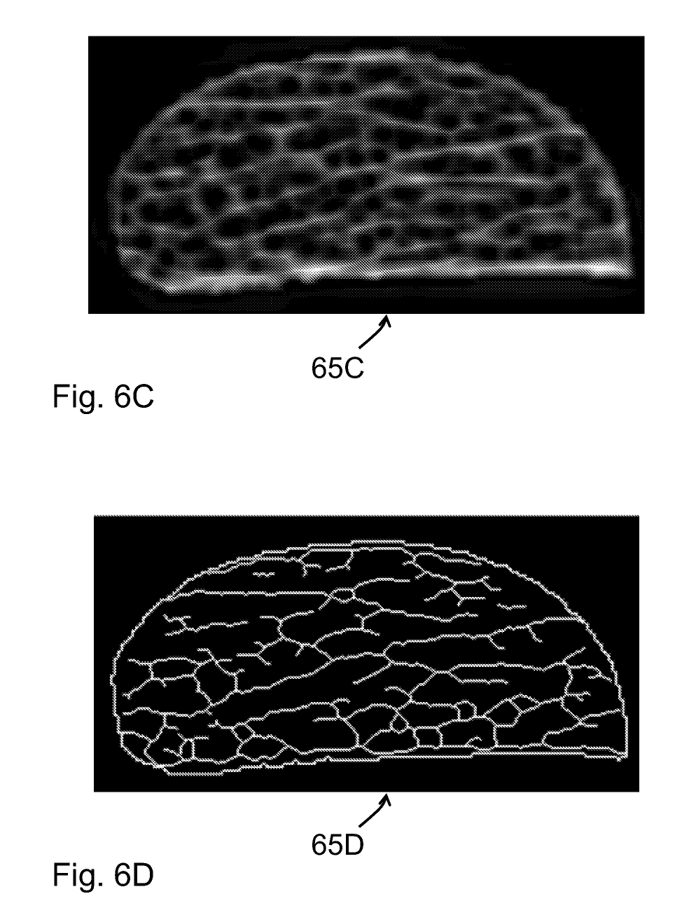

Referring to FIG. 10, in Step 60, a step of segmenting the fibrous structures from the 2D US images is performed. In one embodiment, starting from the in viva 2D US images in B-scan format defined in Step 30 (FIG. 6A), these 2D US images are subjected to a scan conversion producing non linear mappings of the echogenicity, which directly affects the statistics of the echoes, as illustrated at 65A in FIG. 6A.

A method was presented by Prager, R. W., et al., in Decompression and speckle detection for ultrasound images using the homodyned k-distribution, Pattern Recognition Letters, 2003, 24(4-5), pp. 705-713, to derive the approximate unprocessed echo signal envelope from 2D US images. There is proposed a mapping of the form
p=D·ln(I) (2)
where p is the 2D US image, I is the echo envelope signal, and D is the mapping parameter. Theoretically, the intensity values of the echo envelope are known to approximately follow an exponential distribution (Wagner, R. F., et al., Statistics of Speckle in Ultrasound B-Scans, IEEE Transactions on Sonics and Ultrasonics, 1983, 30(3), pp. 156-163). The approach is to match the measured normalized moments <I″>/<I>″ of I in a known B-scan region with the expected values for an exponential distribution that is given by Γ(n+1). <.> is the statistical moment and Γ(.) is the gamma function. This is true for positive values of n, which are not necessarily integers. The algorithm proceeds as follows:
i. Choose an initial value for D;
ii. Invert the compression mapping for a patch of a known B-scan region using the intensity given by I=exp(p/D);
iii. Compute the normalised moments of the intensity data for the powers n 0.25, 0.5, 1.5, 2.0, 2.5, and 3;
iv. Minimize the error vector from the differences between the six normalised moments computed and the normalized moments of an exponential distribution to estimate a value of D.

Subsequently, still in Step 60, the echo envelope intensity 65B of FIG. 6B is modeled using linear acoustics systems (i.e., blind deconvolution) to obtain the image 65C of FIG. 6C. A common approach is the Bamber & Dickinson model (Bamber, J. C. and R. J. Dickinson, Ultrasonic B-scanning: A computer simulation, Phys. Med. Biol., 1980, 25, pp. 463-479). The 2D radio frequency (RF) signal, I_{RF}, is given by:

$${I}_{RF} ( x , y ) = \frac{{\partial }^{2}}{{\partial }^{2} x} H ( x , y ) * Z ( x , y ) + e ( x , y ) \qquad ( 3 )$$

where H is the point spread function (PSF), Z is the 2D acoustic impedance that describes the tissue echogenicity, e is a white Gaussian noise (WGN), and x is the beam axis.

The echo envelope is the Hilbert transform of the 2D RF signal. Thus, it assumes the echo envelope intensity formation model as being quasi-linear and it can be modeled by a 2D convolution equation:
I(x,y)≅h(x,y)*Z(x,y)+e(x,y) (4)
where h is the new PSF system. Thus, the 2D blind deconvolution algorithm can be applied on the echo envelope signal I(x,y) (Taxt, T. and G. V. Frolova, Noise robust one-dimensional blind deconvolution of medical ultrasound images, Ultrasonics, Ferroelectrics and Frequency Control, IEEE Transactions on, 1999, 46(2), pp. 291-299). The solution of the problem is optimum for the following regularization criterion:

$$min ( {‖ h * Z - I ‖}^{2} + {α}_{1} {‖ ∇ ( Z - {Z}_{0} ) ‖}^{2} + {α}_{2} {‖ ∇ ( h - {h}_{0} ) ‖}^{2} ) \\ Z > 0 , h > 0 \qquad ( 5 )$$

where ∇ is the gradient, α₁ and α₂ are two regularization parameters, Z₀(x,y) and h₀(x,y) are the a priori solutions for Z(x,y) and h(x,y). The minimisation of this criterion was done in the Fourier domain.

Still in Step 60, a segmentation of the enhanced images 65C of FIG. 6C is altered using 2D morphological methods as the final post-processing step. It aims to facilitate the extraction of quantitative data concerning fiber bundles (number and cross-sectional area), and also to improve the 2D and 3D visualization of the internal fibrous structure of the fibrous tissue. The 2D morphological operations proceed as follows for each enhanced image:
i. Binarization by Otsu's method, as described by Otsu, N., in A threshold selection method from grey scale histogram, IEEE Transactions on Systems, Man and Cybernetics, 1979, 1(9), p. 62-66, to separate the hyper-echoic structures (interfascicular) from the background;
ii. Dilatation followed by erosion operations;
iii. Thinning operation to reduce all lines to a single pixel thickness, to produce the image 65D of FIG. 6D, in which fibrous structures appear but are not completely defined in the hyper-echoic structures;
iv. WaterShed operation to close the hyper-echoic structures.

The enhanced image 65E of FIG. 6E results from these morphological operations, in which the fibrous structures are closed. If desired, the 2D US images defined in Step 30 are superposed with the enhanced images of FIG. 6E to give the image 65F of FIG. 6F.

As an alternative, in Step 60 a filtering operation is used for enhancement and restoration of coherent structures contained on the ultrasound images 65A (FIG. 6A) of the SDFT. The filtering operation is based on hyperbolic partial differential equation (PDE) types, called shock filter. Shock filter models are based on the idea of creating a sharp shock between two influence zones and producing piecewise constant segmentation. A new parameterized model is presented, which allows a separate control of the enhancement speed and the detection of the shock positions so as to produce an efficient speckles suppression.

According to the shock filtering operation,

$$\frac{\partial I}{\partial t} = - sign ( {G}_{σ} ⊗ {I}_{ηη} ) \vert ∇ I \vert$$

η: direction of gradient

G_{σ}: Gaussian convolution base

[symbol] : convolution operator.

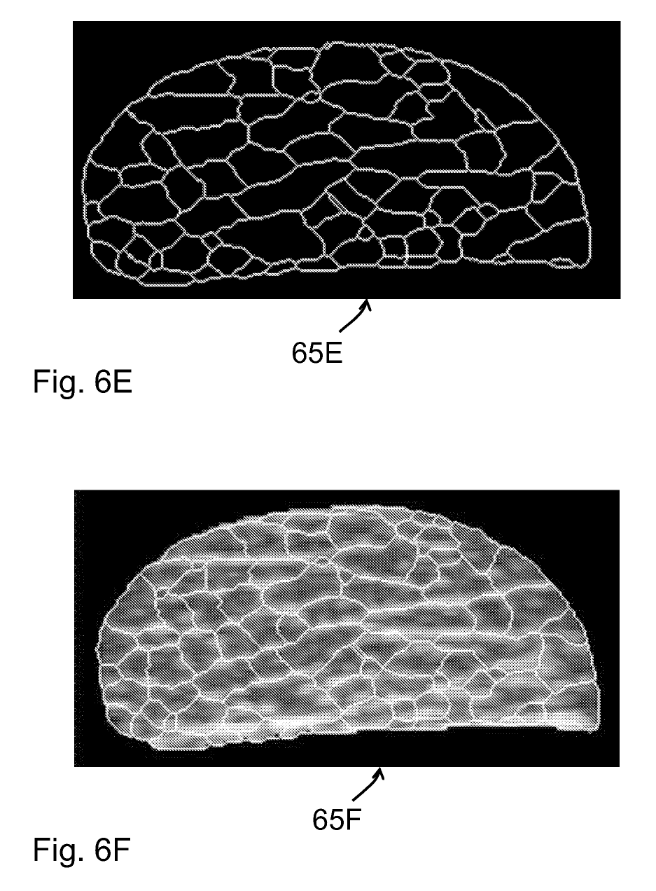

In Step 70, a 3D model showing the fibrous structure of the fibrous tissue is created, and the 3D model 75 is illustrated in FIG. 7. More specifically, the stack of 2D images showing the fibrous structures (FIGS. 6E and 6F) are combined to the external 3D model of Step 50 so as to create the 3D model. The fiber bundle is defined as the smallest closed structure in the images.

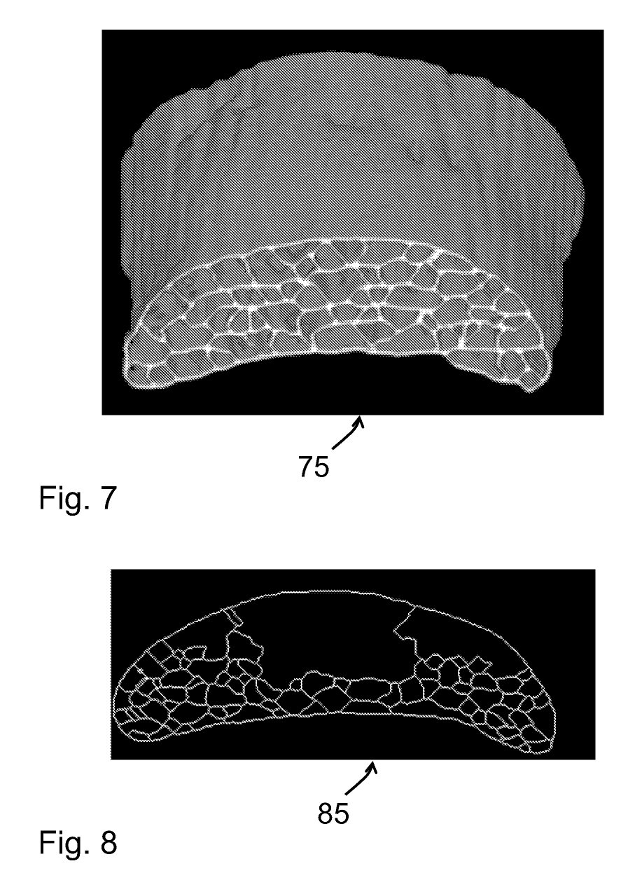

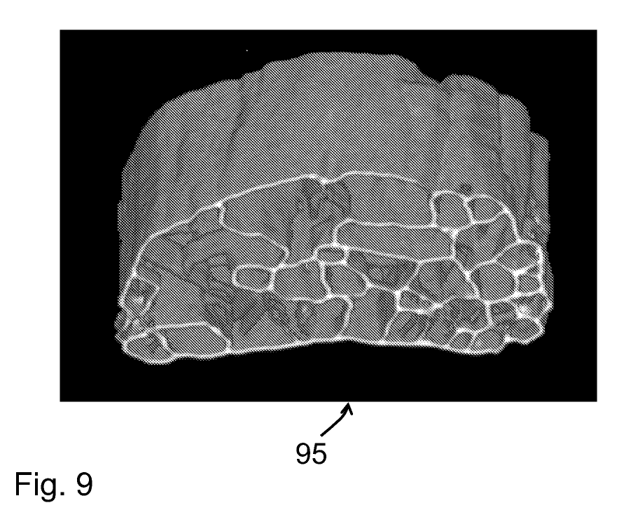

The illustrations of FIGS. 6A-6F and 7 represent a healthy SDFT. In FIG. 8, there is illustrated an injured SOFT 85, in which the biggest closed structure is defined as the injury area. A 3D model 95 of the injured SDFT resulting from the steps of method 10 is shown in FIG. 9, in which the fibrous structure is clearly defined.

The 3D models Statistical analysis was done on the number of fiber bundles and their areas on whole segmented images.

The in vivo 3D US data of a healthy and of an injured SDFT had 114 and 148 frames, respectively. In the case of the injured SDFT, the lesion was located on 30 consecutive 2D US slices. FIGS. 5 and 6 show the segmentation steps of the 2D internal structure of a healthy and of an injured SDFT. The similarity of the structures between the hyper and hypo-echoic zones of the 2D US images and those segmented by the proposed technique can be appreciated.

The quantification of the number of still intact fiber bundles constitutes an information of great value to assess the recovery from injury sustained by the fibrous tissue as it enables the structural integrity of the SDFT to be appreciated.

Density number and area distributions of fiber bundles on a cross section are deduced from the segmented images, which are useful to evaluate the SDFT internal structure. Accordingly, statistical analyses can be performed on the images acquired using the method 10.

### Example

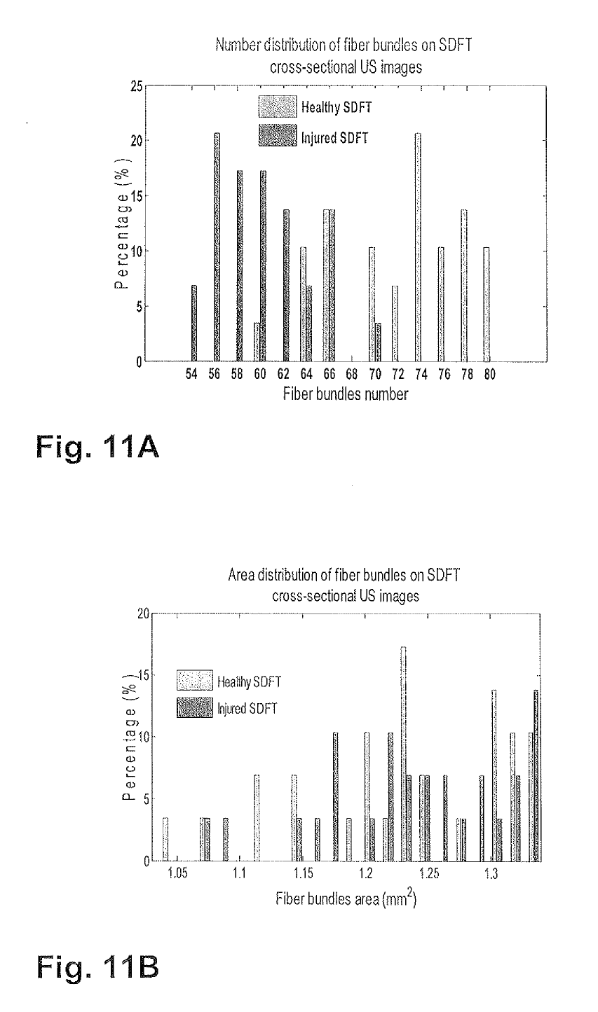

The number of fiber bundles and their areas were deduced on cross-sections. The number of fiber bundles (FIG. 11A), for the whole 2D images, was 73±6 (mean ±standard deviation) for the healthy SDFT, and 61±5 for 30 images where the lesion was located. The areas of fiber bundles (FIG. 8) were 1.23±0.083 mm² for the healthy SDFT and 1.24±0.093 mm² for the pathological case. It is to note that the areas of fiber bundles corroborate those found by Gillis, C. et al. (Effect of maturation and aging on the histomorphometric and biochemical characteristics of equine superficial digital flexor tendon, American Journal of Veterinary Research, 1997, 58(4), pp. 425-430). In this last study, a histomorphometric was used in an in vitro evaluation to deduce SDFT bundle areas in cross-section varying between 0.75-1.41 mm².

The 3D models of FIGS. 7 and 9 allow to evaluate the internal structures of the fibrous tissue (here the SDFT). Thirty successive segmented images of a healthy and of an injured SDFT were used to construct a part of the SDFT volume by using the VolView™ software. Both views allow appreciation of the continuity of fiber bundles, and the 3D characteristics of the lesion. In FIG. 9, the 3D model the injured SDFT emphasizes the injury location in the upper part of the SDFT.

1. A method for producing models of fibrous structure of fibrous tissue, comprising:
obtaining a sequence of two-dimensional ultrasound images along a fibrous tissue;
creating a three-dimensional model of an external portion of the fibrous tissue using the two-dimensional ultrasound images;
segmenting selected fibrous structure data from the two-dimensional ultrasound images; and
creating a three-dimensional model of the fibrous tissue with the fibrous structure by combining the three-dimensional model of the external portion of the fibrous tissue with the selected fibrous structure data.

2. The method according to claim 1, wherein obtaining the sequence of the two-dimensional ultrasound images comprises delimiting an external structure of a selected portion of the fibrous tissue on each said two-dimensional ultrasound image.

3. The method according to claim 2, wherein creating the three-dimensional model of the external portion of the fibrous tissue comprises merging the external structure of each of the two-dimensional ultrasound images.

4. The method according to claim 1, wherein obtaining the sequence of two-dimensional ultrasound images is preformed in free-hand mode.

5. The method according to claim 4, wherein obtaining the sequence of two-dimensional ultrasound images performed in free-hand mode subsequently involves correcting the two-dimensional ultrasound images to reduce an effect of hand movement resulting from the free-hand mode.

6. The method according to claim 5, wherein correcting the two-dimensional ultrasound images is performed using two-dimensional rigid body registration.

7. The method according to claim 1, wherein segmenting selected fibrous structure data from the two-dimensional ultrasound images comprises obtaining non-linear mappings of echogenicity by scan conversion of the two-dimensional ultrasound images.

8. The method according to claim 7, wherein the non-linear mappings are modeled to enhanced images by blind deconvolution.

9. The method according to claim 8, wherein the enhanced images are altered using a two-dimensional morphological operation to define the selected fibrous structure data.

10. The method according to claim 9, wherein the selected fibrous structure data is superposed with a respective one of said two-dimensional ultrasound images.

11. The method according to claim 7, wherein the selected fibrous structure data is superposed with a respective one of said two-dimensional ultrasound images.

12. The method according to claim 7, wherein segmenting selected fibrous structure data from the two-dimensional ultrasound images comprises shock filtering the two-dimensional ultrasound images.

13. The method according to claim 1, further comprising identifying damaged fibrous structures from the selected fibrous structure data.

14. The method according to claim 13, wherein identifying the damaged fibrous structures from the selected fibrous structure data comprises quantifying the damaged fibrous structures as a function of intact fibrous structures to assess a recovery from injury.

15. The method according to claim 1, wherein obtaining the sequence of two-dimensional ultrasound images along the fibrous tissue is performed on an equine superficial digital flexor tendon, and segmenting the selected fibrous structure data from the two-dimensional ultrasound images comprises segmenting fiber bundles.
